(19) United States
(12) Patent Application Publication
(10) Pub. No.: US 2012/0028272 A1
(43) Pub. Date: Feb. 2, 2012

(54) DEVICE AND METHODS FOR ISOLATING CELLS

(75) Inventors: Palaniappan Sethu, Louisville, KY (US); William N. White, Louisville, KY (US); Aman Russom, Louisville, KY (US)

(73) Assignee: UNIVERSITY OF LOUISVILLE RESEARCH FOUNDATION, INC., Louisville, KY (US)

(21) Appl. No.: 13/262,466

(22) PCT Filed: Apr. 1, 2010
(86) PCT No.: PCT/US2010/029663

§ 371 Date: Sep. 30, 2011

Related U.S. Application Data
(60) Provisional application No. 61/165,782, filed on Apr. 1, 2009.

(51) Int. Cl. G01N 33/53; C12N 5/09; C12N 5/078; C12M 1/00; C12N 5/071

(52) U.S. Cl. 435/7.1; 435/283.1; 435/325

(57) ABSTRACT

The invention provides a device and methods for separating and isolating cells.

### CROSS-REFERENCE TO RELATED APPLICATION(S)

This patent application claims the benefit of priority of U.S. application Ser. No. 61/165,782, filed Apr. 1, 2009, which application is herein incorporated by reference.

### BACKGROUND

Tissue from the body can be used for subsequent molecular expression analysis. However, tissue includes heterogeneous mixtures of cells, which greatly impacts the quality of information generated. Sorting of cells into unique sub-populations is essential to obtain high quality information from bodily tissue samples. A commonly biopsied sample is peripheral blood. In addition to transporting oxygen and carbon dioxide, blood is a critical mediator of homeostasis, and its constituents reflect the state of the immune and inflammatory status of the body. Isolation of cells, e.g., nucleated cells, from blood without loss or activation is a challenging task. The functions and significance of nucleated cells (e.g., leukocytes) identified using conventional techniques have been investigated. However, cells present transiently in blood, e.g., in small numbers during certain events, may provide important diagnostic and prognostic information. These cells need to be identified and phenotyped and, if necessary, isolated for subsequent analysis. Currently, none of the leukocyte isolation protocols reliably accomplish isolation of small nucleated cells, comparable in size to erythrocyte debris. Accordingly, devices and methods are needed to isolate populations of cells, such as small nucleated cells.

### SUMMARY OF CERTAIN EMBODIMENTS

Accordingly, as described herein, devices and techniques have been developed for separation of a heterogeneous mixture of cells based on, e.g., size and osmotic properties. The device and technique are useful for isolating specific populations of cells from the heterogeneous mixture of cells. The heterogeneous mixture of cells can be a physiological sample, e.g., blood. The heterogeneous mixture of cells can be from a tissue, the tissue having been dissociated into a solution.

The device can include different modules, e.g., (a) cell quality (e.g., physical, chemical, biological, electrical and magnetic) changing module, e.g., a differential size increase module and/or (b) a cell sorting module, e.g., an inertial focusing module. The differential size increase module can include a channel where a heterogeneous mixture of cells is modified in size via exposure to a hypotonic buffer. Cell size increase is a direct consequence of the imbalance in salt concentration within the cell in the surrounding fluid. The increase in cell size depends on the properties of the cell membrane, extracellular matrix and the presence or absence of aquaporins (water channels). Therefore, when a heterogeneous mixture of cells is subject to a hypotonic buffer, there is a differential increase in size, and in some cases, lysis of certain cell types. Once the size of the cells is differentially increased, the sample can be introduced into the second module. The inertial focusing module can be a spiral device with changing (e.g., increasing) channel width. The aspect ratio of the channels can play an important role in focusing, and for possible channel dimensions attainable using current microfabrication techniques, the smallest focusable particle sizewith currently available technologies is about 4 μm. Cells introduced into the device can be focused based on size as a consequence of balance between inertial forces and Dean forces that develop in curved microfluidic channels, or inertial forces alone that develop in straight channels at high Reynolds number flow. Exposure of small nucleated cells to hypotonic buffer effects a size increase, pushing certain cells size past the minimum focusable limit and secondly the differential size increase allows for more accurate focusing and sorting. Once the cells are sorted, they can exit through different outlets. At each outlet, cells can be returned to isotonic conditions using, e.g., a high salt concentration buffer. This technique is applicable for sorting any heterogeneous mixture of cells including digested tissue and other bodily fluids, e.g., isolation and sorting of leukocytes and other nucleated cells from whole blood.

Certain embodiments of the present invention provide devices and methods that accomplish isolation of cells, e.g., nucleated cells, from biological samples (e.g., blood) and can deliver the cells fractionated into different subpopulations in concentrations appropriate for subsequent processing without the need for additional concentration steps. Erythrocytes may also be eliminated from the biological sample. Certain steps may include the inertial focusing that occurs after blood has been exposed to a hypotonic buffer. This buffer eliminates erythrocytes via lysis and causes an increase in the size of other nucleated cells. Following erythrocyte elimination and differential focusing, the cells can be returned to isotonic conditions via a neutralizing buffer. This technique provides for isolation of nucleated cells (e.g., all nucleated cells), including small nucleated cells, that cannot be isolated using hypotonic lysis alone or those cells that fall below the size limit for reliable focusing using the inertial focusing device.

### BRIEF DESCRIPTION OF THE FIGURES

FIG. 1 illustrates pellets obtained following lysis and centrifugation using microfluidic lysis, in accordance with at least one embodiment.

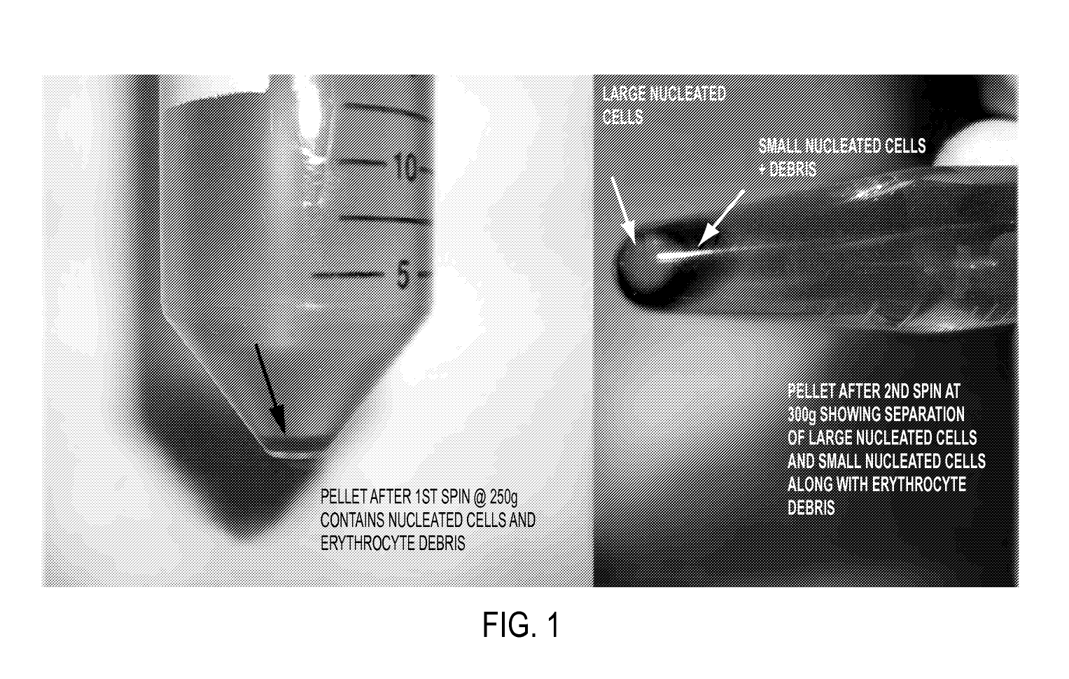

FIGS. 2A, 2B illustrate schematic diagrams of the device, in accordance with at least one embodiment.

FIG. 3 illustrates peripheral blood mononuclear cells (PBMCs) in isotonic conditions and 15 seconds after hypotonic exposure, in accordance with at least one embodiment.

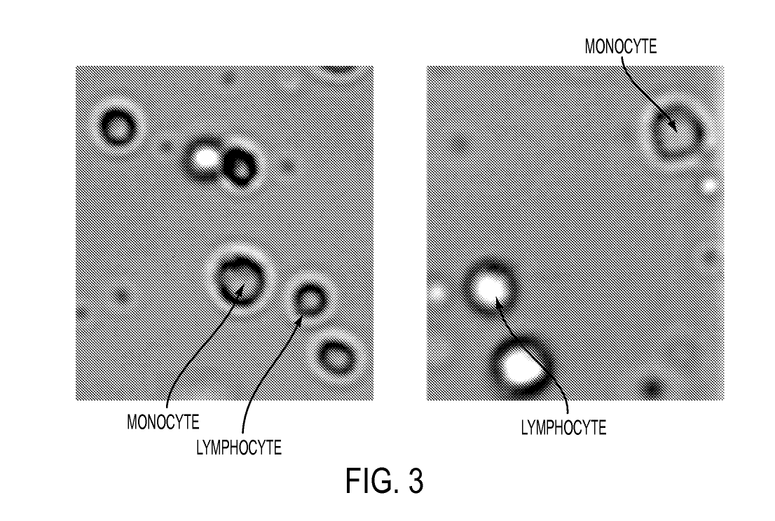

FIG. 4 illustrates granulocytes in isotonic conditions and after hypotonic exposure, in accordance with at least one embodiment.

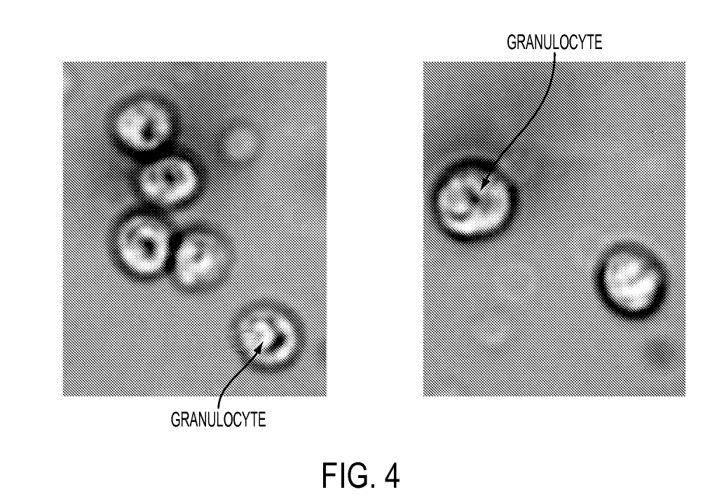

FIGS. 5A-5D illustrates schematic diagrams of aspects of a device, in accordance with at least one embodiment.

FIG. 6 illustrates a chart detailing a minimum focusable particle size as a function of channel aspect ratio, in accordance with at least one embodiment.

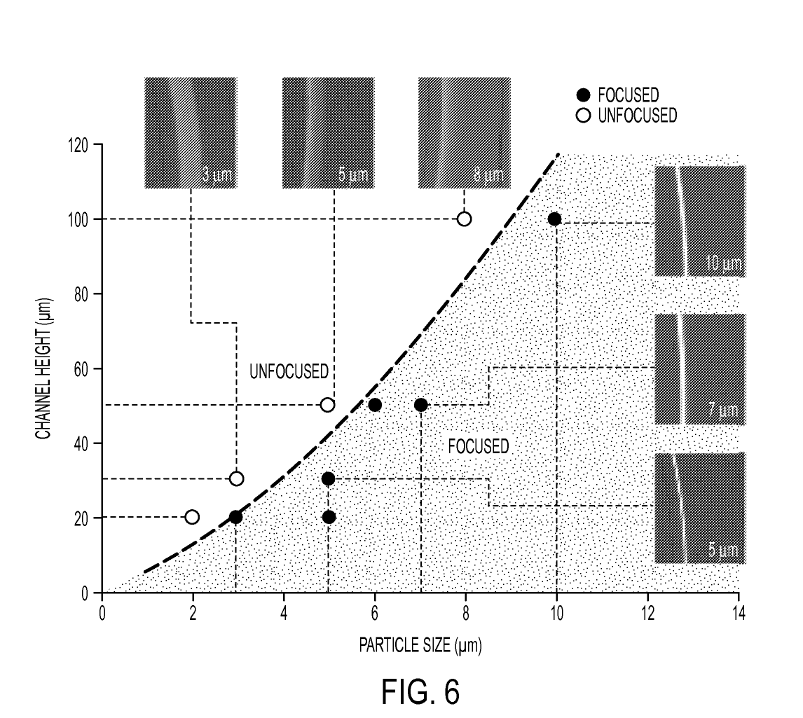

FIG. 7 illustrates a cross-sectional view of a portion of the device including the first module and the second module, in accordance with at least one embodiment.

FIGS. 8A-8G illustrate various cross-sectional shapes of the channel, in accordance with at least one embodiment.

FIG. 9 illustrates a cross-sectional view of a portion of the device including the first module, the second module, and a third module, in accordance with at least one embodiment.

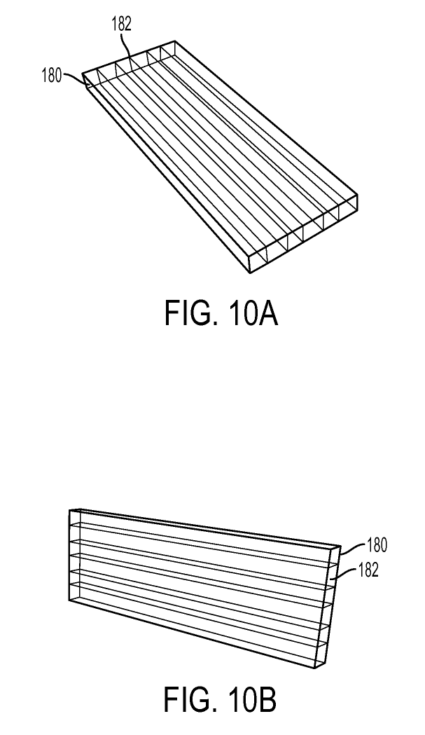

FIGS. 10A-10D illustrate isometric views of two or more fluids for use in the device, in accordance with at least one embodiment.

FIG. 11 illustrates the increase in cell size following exposure to hypotonic solution.

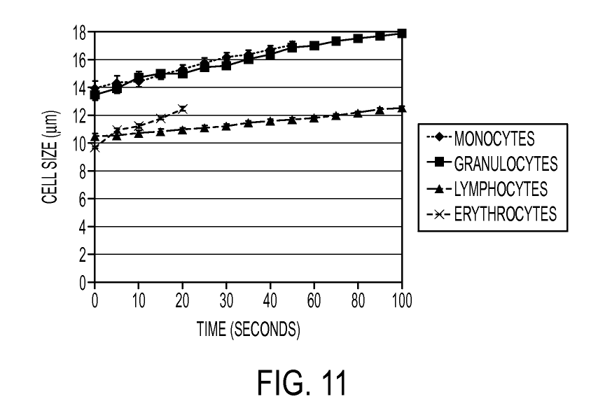

### DETAILED DESCRIPTION

Described herein is a device that performs sorting and isolation of different cell types from a heterogeneous mixture. The device can perform the following functions, among others: (a) use a hypotonic buffer to simultaneously eliminate certain cell types and cause a cell type specific size increase in other nucleated cells, (b) use inertial focusing to push cell size past minimum focusable size and sort inflated nucleated cells based on size (c) return cells to isotonic conditions and to their original size following sorting and optional erythrocyte elimination.

Certain embodiments provide a microfluidic device, comprising a first module that is a differential size increase module and a second module that is an inertial focusing module.

In certain embodiments, the device may further comprise a third module 194 that comprises molecules for binding mRNA or protein. In certain embodiments, the third module comprises molecules for binding mRNA. In certain embodiments, the third module comprises molecules for binding protein.

In certain embodiments, the third module comprises a column or channel that comprises beads for mRNA capture.

Certain embodiments provide methods for isolating nucleated cells from a sample that comprises a population of cells, comprising processing the sample using a microfluidic device as described herein so as to isolate nucleated cells from the sample.

In certain embodiments, the nucleated cells comprise CD31+/CD61+ cells.

In certain embodiments, the nucleated cells comprise CD146+/CD61+ cells.

In certain embodiments, the nucleated cells comprise endothelial cells.

In certain embodiments, the nucleated cells comprise tumor cells.

In certain embodiments, the methods may further comprise identifying the nucleated cells.

In certain embodiments, the nucleated cells are identified using at least one antibody.

Samples obtained from this device can be used, e.g., directly without any additional processing for, e.g., flow cytometry, cellomics studies, genomics studies, proteomic studies or metabolomic studies.

Certain embodiments provide a fluidic device comprising: a first module including a cell quality changing module; a second module including a cell sorting module having at least one channel; and the first module and the second module are fluidly coupled with one another.

In certain embodiments, the first module is coupled with a first side of a substrate and the second module is coupled with a second side of the substrate, the substrate including a conduit coupled between a first module outlet and a second module inlet.

In certain embodiments, the device further comprises a magnetic field disposed adjacent to the cell sorting module.

In certain embodiments, the device further comprises an electrical field disposed adjacent to the cell sorting module.

In certain embodiments, the device further comprises a coating along an inner wall of a channel of the cell sorting module.

In certain embodiments, the cell sorting module includes at least one channel, the at least one channel has a non-symmetrical cross-section.

In certain embodiments, the cell sorting module includes at least one channel, two or more fluids disposed within the channel.

In certain embodiments, the two or more fluids include a first fluid and a second fluid, the first fluid has a different property than the second fluid.

In certain embodiments, the different property relates to one or more of magnetic properties, electrical properties, density, or combinations thereof.

In certain embodiments, the first fluid has a different density than the second fluid.

In certain embodiments, the first fluid is disposed directly adjacent the second fluid.

In certain embodiments, the first fluid is layered vertically or horizontally or combinations thereof relative to the second fluid.

In certain embodiments, the first fluid is co-axial with the second fluid.

In certain embodiments, the cell sorting module includes at least one channel, the at least one channel has a triangular, semispherical, quarter spherical cross-section, or combinations thereof.

In certain embodiments, the device further comprises one or more filters disposed within a channel of the sorting module.

In certain embodiments, the sorting module includes at least one channel, the at least one channel includes non-constant channel width along a portion of the channel length.

In certain embodiments, the device further comprises a third module, the third module including molecules that bind mRNA or protein.

In certain embodiments, the second module channel is a spiral channel.

In certain embodiments, the second module further includes a sample inlet and a solution inlet.

In certain embodiments, the second module further includes at least one outlet.

Certain embodiments provide a method comprising: isolating at least one population of cells from a sample with a microfluidic device described herein, where isolating cells includes flowing cells through a cell quality changing module; and flowing cells through a cell sorting module.

In certain embodiments, the at least one population of cells that is isolated is a population of nucleated cells.

In certain embodiments, the at least one population of cells that is isolated is a population of leukocytes.

In certain embodiments, the at least one population of cells that is isolated is a population of lymphocytes, monocytes or granulocytes.

In certain embodiments, the nucleated cells comprise CD31+/CD61+ cells.

In certain embodiments, the nucleated cells comprise CD146+/CD61+ cells.

In certain embodiments, nucleated cells comprise endothelial cells.

In certain embodiments, nucleated cells comprise tumor cells.

In certain embodiments, the method further comprises identifying the at least one population of cells.

In certain embodiments, the cells are identified using at least one antibody.

In certain embodiments, the sample is a physiological sample.

In certain embodiments, the physiological sample is a tissue sample that has been dissociated into solution.

In certain embodiments, the sample is a blood sample.

In certain embodiments, the method further comprises exposing the sample to a hypotonic buffer while in the device.

In certain embodiments, the method further comprises returning the at least one population of cells that has been isolated to an isotonic condition.

A mechanism of cell lysis/elimination is hypotonic shock. For example, erythrocytes are extremely sensitive to tonicity of the solution and lyse in about 10-15 seconds. A blood sample that is used can be mixed in ratios between 1:20-1:50 based on erythrocyte concentration with a hypotonic buffer, which results is lysis of erythrocytes and swelling of nucleated cells.

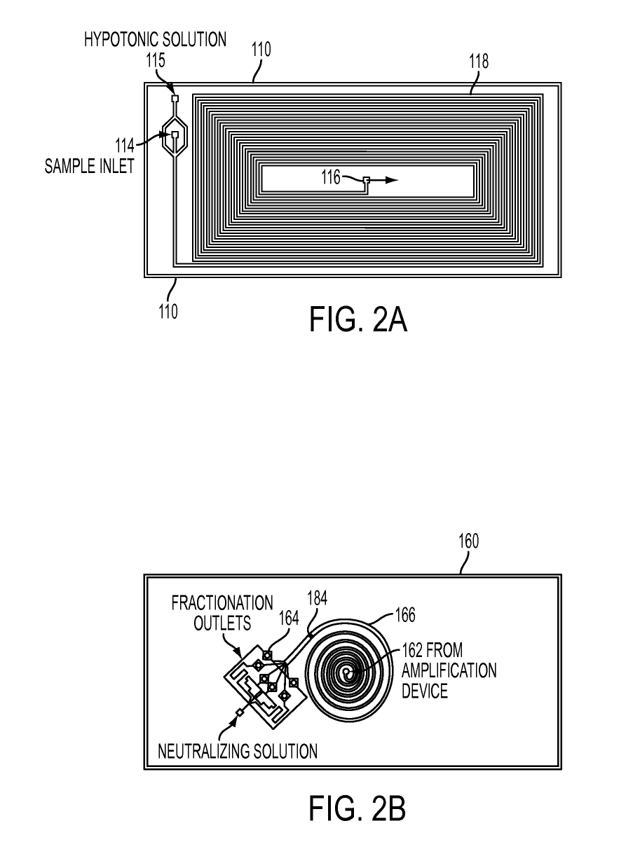

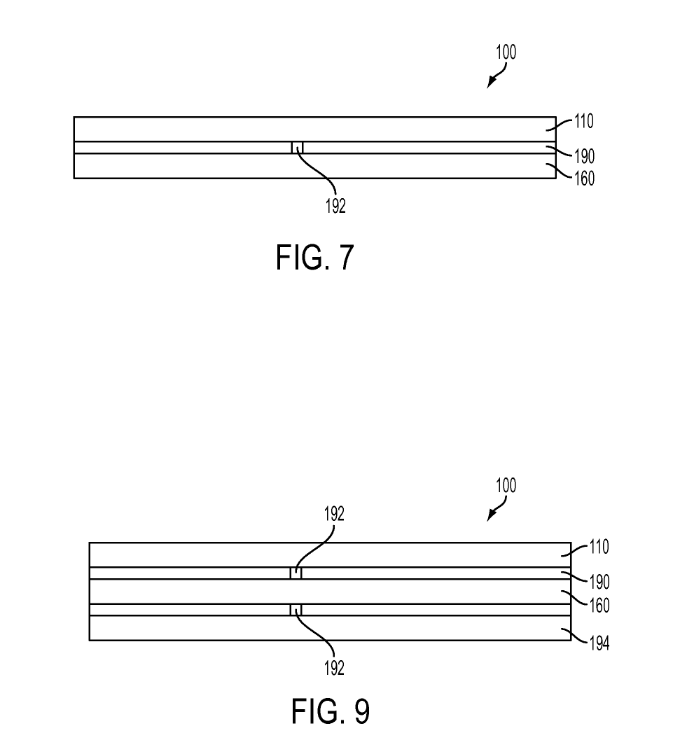

A fluidic device 100 is illustrated in FIGS. 2A, 2B, 7. The fluidic device 100 includes a first module 110, such as cell property charging module 112. In an option, the cell property changing module includes a cell size amplification module. Additionally, the density, stiffness, shape etc. can also be altered in this module. For example, the size can be modified by binding two or more cells together, binding cells to beads, binding cells to particles, or binding large molecules to cells. Other options can be used to change cell size include, but are not limited to changing salt concentration (osmosis), changing temperature, or changing pH levels. Certain cells may swell at different rate for certain temperatures, salt concentration, or pH levels. In a further option, binding various chemical or biochemical signs, or the presents thereof, can be exploited to differentially increase or decrease cell size or other properties. Examples include, but are not limited to detergents, lysis agents, sugars, alcohol, growth factors, chemokines, cytokines, antibodies that affect cell size and conformation, or combinations thereof. For instance, in certain embodiments, a channel 166 of the device 100 comprises antibodies bound to the channel. Various cell size amplification techniques, such as those discussed above, can be used alone or in combination to optimize the result.

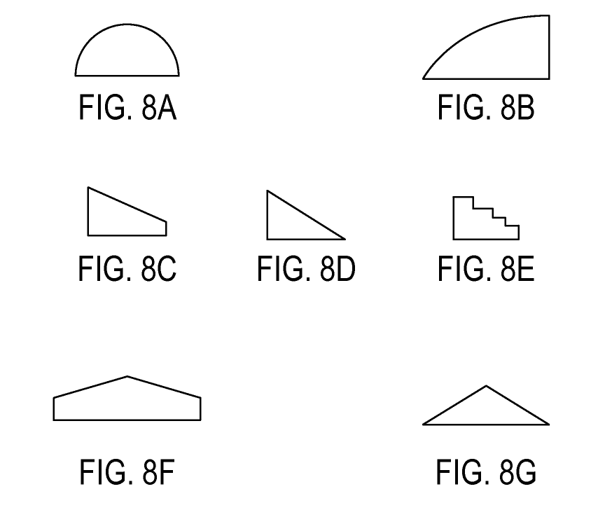

Referring again to FIGS. 2A, 2B, and 7, the first module 110 includes at least one inlet 114, such as a sample inlet, at least one outlet 116, and one or more channels 118 therebetween. In a further option, the first module 110 includes at least one inlet 115, such as a hypotonic solution inlet. The fluidic device 100 further includes a second module 160 fluidly coupled with the first module 110. In an option, the fluidic device 100 further includes a substrate 190 disposed between the first module 110 and the second module 160, and the first module 110 and the second module 160 are coupled with the substrate 190, for example by bonding, molding, spray-on coating, or combinations thereof The channels 118, 166 can be formed, for example, using soft lithography techniques. For instance, a layer of photo resist can be patterned on a side of a substrate, and a polymer can be cast on to replicate the channels. The resulting polymer mold can be bound with the substrate. For more complex shapes, as discussed hereafter, the photolithography can be used to form layers of changing shapes to achieve more complex channel shapes, such as those shown in FIGS. 8A-8G.

The substrate 190, in an option, includes at least one conduit 192 therein. In an option, the conduit 192 fluidly couples at least one outlet 116 of the first module 110 with the inlet of the second module 160.

The second module 160, such as a cell sorting module, includes at least one inlet 162, at least one outlet 164, and one or more channels 166 therebetween. Multiple outlets 164 (e.g., 2, 3, 4, 5, 6, 7, 8 or 9 outlets) can be used for example, to collect different cells therefrom. The one or more channels 166 form a conduit between the inlet 162 and the outlet 164.

In a further option, the cell sorting module operates to sort a sample of cells to select a subset of cells, for instance, by exploiting secondary properties or a combination of secondary properties of the cell including, but not limited to, density and sedimentation, shape, size, electrical properties, such as, but not limited to electrophoretic, dielectrophoretic, magnetic, such as, but not limited to, ferromagnetic, diamagnetic, paramagnetic, chemotatic (i.e. attraction to a particular stimulus and movement towards that stimulus), or chemo-repellent (i.e. repulsion to a particular stimulus and movement away from that stimulus), or combinations thereof. For example, an external electrical or magnetic field can be applied or be disposed adjacent the cell sorting module which affects the flow and/or property of the cells, or a subset of the sample. These can be used with above and below examples of cell sorting modules.

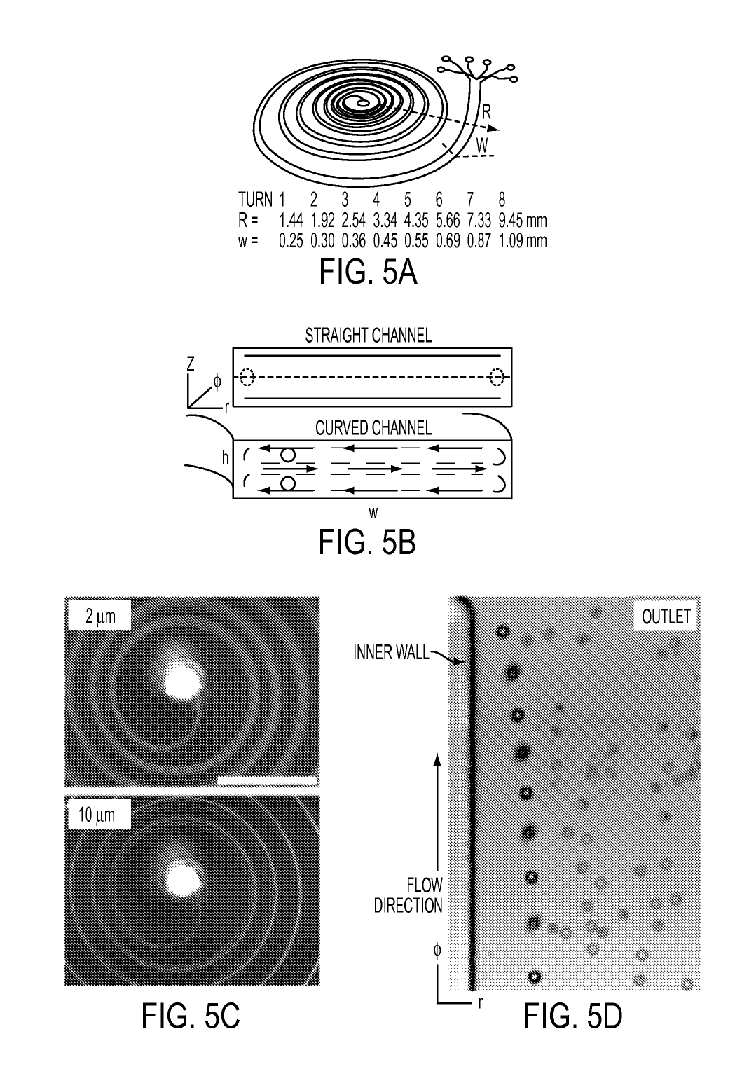

FIGS. 5A-5D illustrates an example schematic diagram of aspects of the device 100. FIG. 5A shows spiral device with a footprint of 2.1 cm in diameter. FIG. 5B shows schematic particle focusing points along the height of the channel (z-plane) for straight rectangular and curved channels, and FIG. 5C shows a fluorescence image of particle mixture (2 and 10 mm) flowing at a flow rate of 2 mL/min. Scale bar: 2 mm. Lastly, FIG. 5D shows an image at the outlet showing self-ordered, longitudinally alternate positioned particle trains of 10 mm particles. The scattered 2 μm particles are highlighted for clarity. Scale bar: 50 mm.

In an option one or more fluids are disposed within the channels 118 and/or channels 166. In an option, a coating is disposed along an inner wall of a channel of the cell sorting module, allowing for certain cells to bind to the coating as fluid flows through the channel 166. In a further option, two or more fluids are disposed within the channels 118, 166. In another option, the two or more fluids include a first fluid 180 and a second fluid 182. The two or more fluids can be used to sort the cells of the sample.

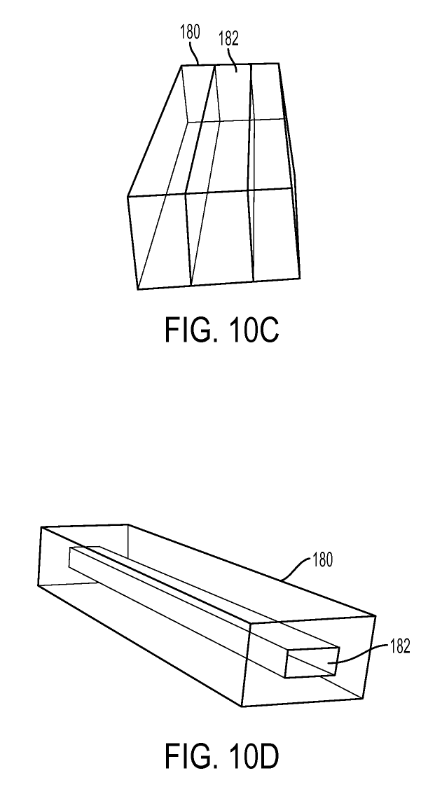

The first fluid 180 and the second fluid 182 are, in an option, disposed directly adjacent to one another. The first fluid 180 can further be layered within the channels 118, 166, for instance as shown in FIGS. 10A-D. For instance, the first fluid 180 and the second fluid 182 are layered horizontally within the channels 118, 166. In another option, the first fluid 180 and the second fluid 182 are layered vertically within the channels 118, 116. In yet a further option, the first fluid 180 is coaxial with the second fluid 182. The layering of fluids can be used, for instance with fluids that differ from one another.

In a further option, the first fluid 180 and a second fluid 182, in an option, have different properties from one another, and a mismatched. In an option, the different property relates to one or more of magnetic properties, electrical properties, density, composition, or combinations thereof. In yet another option, the first fluid 180 has a different cell size than the second fluid 182.

Using the layers and/or different properties of the first fluid 180 and the second fluid 182 allows for the sorting process to occur. For instance, the cell size amplification module can be used to decrease or increase the cell size in the sample of fluid. The channel 166 can then be used to sort based on the cell size. For instance, the channel 166 can have a filter 184 that only permits a cell having a certain size therethrough. In another option, the channel 166 can have a variety of cross-sectional shapes, as shown in FIGS. 8A-8G to further enhance the sorting process. In a further option, the first fluid 180 can have magnetic properties, and can attract certain properties of the second fluid. For example, the cross-sectional shape can include a non-symmetrical cross-section, a triangular, semispherical, quarter spherical cross-section, or combinations thereof.

In an option, the second module 160 includes, for example, a spiral shaped channel 166 that includes for example, two geometrical features that result in particle focusing: high-aspect-ratio rectangular cross-section geometry and curvature in the form of a spiral with channel width increasing with the radius of curvature of the channel 166. Focusing based on size is accomplished due to dominant lift and Dean forces. In addition to lift forces, flows through curved channels 166 introduce Dean forces due to a secondary cross-sectional flow (Dean Flow) and the balance between these forces causes focusing (see FIG. 2B).

Sorting via focusing occurs when the flow rate results in a Reynolds number of 100 or greater, where the flow is defined as being in the transition regime between laminar and turbulent flow. Expanding channel width along a length of the channel 166 (i.e. the channel 166 has a non-constant channel width) can enhance the sorting by causing amplification of the separation resulting in the widening of streamlines.

The device 100 can be made using standard microfabrication techniques like soft-lithography, polymer molding, casting or glass or silicon micromachining. The device itself can include an inlet channel(s) for sample and hypotonic solution. The two can mix at predetermined ratios using, e.g., a double herringbone architecture at the bottom of the channels and follow a radially inward path to an outlet that is located, e.g., at the center of the substrate. A conduit 192 can be drilled at that location to transfer fluids to the other side of the substrate to the sorting device, as shown in FIG. 7. See, e.g., Sethu et al. (Anal. Chem., 76, 6247-6253 (2004)).

The spiral inertial sorting device can be located on the other side of the substrate and can also be fabricated using a variety of microfabrication techniques described herein. The flow can be radially outward. At the exit, the channel can split into several (e.g., 2, 3, 4) outlets to accomplish fractionation. Each channel can also be connected to a 10× buffer to accomplish return of nucleated cells to isotonic conditions.

In a further option, migration of the cells can be dictated by forces include inertial lift forces. In another option, the migration of forces is dictated by Dean's forces, such as for spiral or curved channels.

Cells focused and isolated using this technique include small nucleated cells present, e.g., in tissue and blood, that are typically lost using conventional lysis or density gradient techniques and microfluidic lysis due to their size and tendency to pellet with cell debris during centrifugation in the multiple wash steps involved using these techniques. Such cells are not sorted using inertial size based focusing techniques alone, as their size falls below the cutoff of about 4 μm, which is the minimum particle/cell size for such sorters based on experimental evaluation.

Thus, sorting can occur following exposure to hypotonic buffer. Apart from causing elimination of certain cell types, this also results in a cell-type specific increase in the size of nucleated cells. Small nucleated cells, typically in the size range of about 3-5 μm, which pellet with erythrocyte debris during centrifugation or fall below the size cutoff of inertial focusing based sorters, increase in size from about 3-4 μm to about 5-6 μm in diameter, enabling inertial focusing and isolation.

Other larger circulating nucleated cells also undergo a change in size and size-based sorting allows fractionation of cells based on their hypotonic exposure related size.

Prior to exiting the device, the cells can be returned to isotonic conditions, which also causes these cells to return to their original sizes. In a further option, the sample can be re-circulated through the same device or a different device haven't different cell sorting properties using the techniques discussed herein.

### Sorting and Isolation of Nucleated Cells from Whole Blood

Blood is a living tissue composed of blood cells suspended in plasma. Plasma, which comprises 55% of blood fluid, is mostly water (90% by volume), and contains dissolved proteins, glucose, mineral ions, hormones, carbon dioxide (plasma being the main medium for excretory product transportation), platelets and blood cells themselves. The blood cells present in blood are mainly red blood cells (also called RBCs or erythrocytes) and white blood cells, including leukocytes and platelets (also called thrombocytes). Erythrocytes are anucleated cells that primarily serve in oxygen transport and delivery to tissue make up the majority of the cellular fraction in blood and outnumber other nucleated cells by approximately 5000:1. There exist other nucleated cellular populations in blood small numbers that have not been identified or accurately phenotyped due a variety of factors. Accurate enumeration of nucleated cells in blood and subsequent molecular and cellular analysis often requires depletion or removal of erythrocytes. The highest yield of nucleated cells can be accomplished using a microfluidic lysis protocol subjecting blood to a controlled exposure to deionized water (Sethu et al., Anal. Chem., 78, 5453-5461 (2006)). This exposure preferentially causes rapid lysis of erythrocytes and has a minimal effect on other nucleated cells as long as the cells are returned to isotonic conditions within 15 seconds. Though this technique provides the highest yield of nucleated cells with minimal activation, several small nucleated cells, many of which are yet to be phenotyped, are lost during subsequent centrifugation based purification steps.

As described herein, a device has been developed that uses controlled exposure to hypotonic solutions with, e.g., simultaneously, inertial and geometric focusing to obtain depletion of erythrocytes and sorting of various nucleated populations in blood. The sorting occurs when blood is exposed to the hypotonic buffer. During this time, nucleated cells increase in size, allowing differential sorting from erythrocyte debris. This device therefore can accomplish simultaneous erythrocyte depletion, nucleated cell size amplification for sorting, sorting for cell sorting into different subpopulations based on size, return to isotonic conditions with or without cell fixation and final delivery of sample in concentrations suitable for subsequent cellomics, genomics, proteomics and metabalomics. Cells obtained via this device include cells identified via conventional techniques including various leukocyte sub-populations, cells present in small numbers including circulating dendritic cells, fibrocytes and endothelial cells, other nucleated cells including hematopoietic stem cells, mesenchymal stem cells, other progenitor, precursor and nucleated cells that exist in blood but are yet to be phenotyped.

The fluidic device 100 will now be illustrated by the following non-limiting Examples.

### EXAMPLE 1

### Erythrocyte Elimination

A first aspect of a device of the invention is the ability of the device to function to eliminate erythrocytes, as erythrocytes can have little valuable information for subsequent downstream analysis. Certain hematopoietic stem cells on maturity lose their nucleus to become reticulocytes, which then enter circulation and lose organelles to become mature erythrocytes. Erythrocytes are also characterized by the absence of an interior cytoskeletal network and are characterized by a cortical cytoskeleton of spectrin and ankyrin. This causes erythrocytes to become extremely flexible, enabling passage through narrow capillaries in circulation. Due to the absence of an interior cytoskeletal network, the kinetics of erythrocyte response to hypotonic solutions is distinctly different from other circulating nucleated cells. This phenomenon is exploited to selectively deplete erythrocytes from other nucleated cells. Erythrocytes typically lyse in about 8-15 seconds, whereas nucleated cells require more than 40 seconds to lyse. Selective depletion of erythrocyte can be accomplished in the device in a highly controlled fashion. A stream of blood can be exposed to a hypotonic solution in ratios that can vary from about 10:1 to 60:1 (hypotonic solution:blood) based on the type of solution and concentration of cells in blood. Using the device, the stream of blood is exposed to the hypotonic solution for a period of about 10 seconds, sufficient for erythrocyte lysis, and then returned to isotonic conditions via introduction of a 10× buffer.

### EXAMPLE 2

### Size Amplification of Nucleated Cells

The cutoff for reliable inertial sorting is about 4 μm for various geometries/aspect ratios of the sorting channel. Leukocytes greater than 8 μm can be focused reliably using this device. Other small nucleated cells, e.g., cells typically lost with erythrocyte debris during centrifugation, are not isolated using the inertial sorting device. In order to accomplish sorting and subsequent isolation of the small nucleated cells, the size of the cells first can be increased. This can be accomplished by exposure to a hypotonic buffer. Secondly, leukocytes are typically in the size range of about 10-15 μm and need a larger difference in size for efficient isolation. This also happens on exposure to hypotonic solution as each cell swells based on its membrane, extracellular matrix and presence/absence of aquaporins or water channels. This differential size increase ensures efficient sorting and separation.

### EXAMPLE 3

### Inertial Size Based Focusing of Cells in a Spiral Channel

Once erythrocyte elimination and differential size amplification has been accomplished, the cells can enter the focusing module of the device. At Reynolds numbers greater than 100, Dean's forces play a significant role in causing fluid rotation within the channel, which also causes ordering of cells and particles based on size. In addition to high aspect ratio channels and spiral geometry, the channels may also have a widening cross-section as the channel progresses. This ensures amplification of focused cells for efficient isolation.

### EXAMPLE 4

### Return to Isotonic Conditions and Collection

Following size based focusing in hypotonic buffer; cells can be collected via different outlets. Prior to exiting the outlet channels, the cells can be returned to isotonic conditions via exposure to a high salt concentration solution. This ensures that cells are exposed to the hypotonic conditions for about 10-15 seconds, which is the minimum time required for sorting. This prevents cell activation due to prolonged exposure to hypotonic solutions.

### EXAMPLE 5

### Additional Components for Blood mRNA/Protein Isolation

The device can include an mRNA isolation module. Blood flows through the initial lysis/cell inflation device and erythrocyte elimination is accomplished. Leukocytes size increases to enable size based sorting using the second module. The mRNA isolation module can use leukocyte lysis buffer instead of 10× buffer that is used to return leukocytes to isotonic conditions. This buffer causes lysis of leukocytes following sorting from erythrocyte debris. Released leukocyte cellular contents then can flow through a column or channel packed with beads for mRNA capture. Once the entire sample has flowed through 3 modules, the beads are isolated for subsequent processing and amplification. This capture technology can also be modified to capture proteins, e.g., intracellular proteins.

### EXAMPLE 6

### Direct Leukocyte Capture Using Immuno-Modified Gold Coated Surfaces

Leukocytes are an extremely heterogeneous population. Recent studies have shown that purified leukocyte subpopulations can provide identification of unique expression profiles not attainable using whole leukocyte studies. A high efficiency immuno-affinity cell capture device can be used to isolate specific leukocyte subpopulation directly from whole blood. This technology can use polyethylene glycol (PEG)-thiol chemistry to immobilize NH₂ groups on the floor of a channel coated with a thin layer of gold. Specific antibodies will then be immobilized to the NH groups to achieve an immuno-modified surface. This device can operate in a continuous flow through fashion for rapid processing. Blood can be introduced into the device and cells specific to the antibody will be captured, while other cells will be flushed out of the channel. The height of the channel can be restricted to about 20 μm to ensure that the cells interact with the floor of the channel, increasing the probability of capture. This process can use OPSS-PEG-NH groups, which on hydrolysis yield a SH (thiol) group that affixes to gold surfaces. A PEG-OPSS group may be used to minimize non specific binding. The device can operate under slow continuous flows as well as faster pulsatile flows.

While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fluidic device comprising:
a first module including a cell quality changing module;
a second module including a cell sorting module having at least one channel; and
the first module and the second module are fluidly coupled with one another.

2. The fluidic device as recited in claim 1, wherein the first module is coupled with a first side of a substrate and the second module is coupled with a second side of the substrate, the substrate including a conduit coupled between a first module outlet and a second module inlet.

3. The fluidic device as recited in claim 1, further comprising a magnetic field disposed adjacent to the cell sorting module.

4. The fluidic device as recited in claim 1, further comprising an electrical field disposed adjacent to the cell sorting module.

5. The fluidic device as recited in claim 1, further comprising a coating along an inner wall of a channel of the cell sorting module.

6. The fluidic device as recited in claim 1, wherein the cell sorting module includes at least one channel, the at least one channel has a non-symmetrical cross-section.

7. The fluidic device as recited in claim 1, wherein the cell sorting module includes at least one channel, two or more fluids disposed within the channel.

8. The fluidic device as recited in claim 7, wherein the two or more fluids include a first fluid and a second fluid, the first fluid having a different property than the second fluid.

9. The fluidic device as recited in claim 8, wherein the different property relates to one or more of magnetic properties, electrical properties, density, or combinations thereof.

10. The fluidic device as recited in claim 8, wherein the first fluid has a different density than the second fluid.

11. The fluidic device as recited in claim 8, wherein the first fluid is disposed directly adjacent the second fluid.

12. The fluidic device as recited in claim 8, wherein the first fluid is layered vertically or horizontally or combinations thereof relative to the second fluid.

13. The fluidic device as recited in claim 8, wherein the first fluid is co-axial with the second fluid.

14. The fluidic device as recited in claim 1, wherein the cell sorting module includes at least one channel, the at least one channel has a triangular, semispherical, quarter spherical cross-section, or combinations thereof.

15. The fluidic device as recited in claim 1, further comprising one or more filters disposed within a channel of the sorting module.

16. The fluidic device as recited in claim 1 wherein the sorting module includes at least one channel, the at least one channel includes non-constant channel width along a portion of the channel length.

17. The fluidic device as recited in claim 1, further comprising a third module, the third module including molecules that bind mRNA or protein.

18. The fluidic device as recited in claim 1, wherein the second module channel is a spiral channel.

19. The fluidic device as recited in claim 1, wherein the second module further includes a sample inlet and a solution inlet.

20. The fluidic device as recited in claim 1, wherein the second module further includes at least one outlet.

21. A method comprising:
isolating at least one population of cells from a sample with a microfluidic device of claim 1, where isolating cells includes flowing cells through a cell quality changing module; and
flowing cells through a cell sorting module.

22. The method as recited in claim 21, wherein the at least one population of cells that is isolated is a population of nucleated cells.

23. The method as recited in claim 21, wherein the at least one population of cells that is isolated is a population of leukocytes.

24. The method as recited in claim 21, wherein the at least one population of cells that is isolated is a population of lymphocytes, monocytes or granulocytes.

25. The method as recited in claim 22, wherein the nucleated cells comprise CD31+/CD61+ cells.

26. The method as recited in claim 22, wherein the nucleated cells comprise CD146+/CD61+ cells.

27. The method as recited in claim 22, wherein the nucleated cells comprise endothelial cells.

28. The method as recited in claim 22, wherein the nucleated cells comprise tumor cells.

29. The method as recited in claim 21, further comprising identifying the at least one population of cells.

30. The method as recited in claim 29, wherein the cells are identified using at least one antibody.

31. The method as recited in claim 21, wherein the sample is a physiological sample.

32. The method as recited in claim 31, wherein the physiological sample is a tissue sample that has been dissociated into solution.

33. The method as recited in claim 21, wherein the sample is a blood sample.

34. The method as recited in claim 21, comprising exposing the sample to a hypotonic buffer while in the device.

35. The method as recited in claim 21, further comprising returning the at least one population of cells that has been isolated to an isotonic condition.
